(19) United States
(12) Patent Application Publication
(10) Pub. No.: US 2023/0248785 A1
(43) Pub. Date: Aug. 10, 2023

(54) METHODS AND COMPOSITIONS FOR TREATING METABOLIC CONDITIONS

(71) Applicant: BACTANA CORP., Farmington, CT (US)

(72) Inventors: John A. KALLASSY, West Hartford, CT (US); Simon A. MCMANUS, West Hartford, CT (US); Emily GAGNON, Colchester, CT (US)

(21) Appl. No.: 18/161,617

(22) Filed: Jan. 30, 2023

Related U.S. Application Data
(63) Continuation-in-part of application No. PCT/US21/43971, filed on Jul. 30, 2021.
(60) Provisional application No. 63/059,130, filed on Jul. 30, 2020.

(51) Int. Cl. A61K 35/741; C12N 1/20; A61P 3/10

(52) U.S. Cl. CPC A61K 35/741; C12N 1/20; A61P 3/10

(57) ABSTRACT

The disclosure provides methods for treating or mitigating a disease or condition characterized by impaired glucose metabolism comprising administering an effective amount of Faecalibacterium prausnitzii, or a composition made from a culture of Faecalibacterium prausnitzii, to a subject in need thereof; together with compositions useful in said method, and methods of making the same.

### PRIOR RELATED APPLICATIONS

This application is a continuation-in-part, filed under 35 U.S.C. § 111, of PCT Application No. PCT/US2021/43971, filed on Jul. 30, 2021, which claims priority to, and the benefit of U.S. Provisional Application No. 63/059,130, filed on Jul. 30, 2020, the entire contents of which are incorporated herein by reference.

### FIELD

The disclosure relates to compositions and methods for treating a disease or condition characterized by impaired glucose metabolism in humans and animals using Faecalibacterium prausnitzii probiotics or compositions made from culture of Faecalibacterium prausnitzii.

### BACKGROUND

Insulin is a peptide hormone produced by β-cells of the pancreatic islets. When blood sugar levels rise, the β-cells release insulin, which has a variety of important effects on metabolism, promoting absorption of glucose from the blood into liver, fat and skeletal muscle cells, thereby reducing the blood sugar levels, and triggering the production of glycogen (glycogenesis) and/or fats (lipogenesis), depending on the type of cell, as well as inhibiting the production of glucose by the liver. Impaired glucose metabolism can be a function of inadequate insulin production by the β-cells, inadequate response of cells to insulin (insulin resistance), or both.

Type 1 diabetes, also known as juvenile diabetes or insulin-dependent diabetes, is a chronic condition in which the pancreas produces little or no insulin, so patients require exogenous insulin. Type 1 diabetics may also develop insulin resistance, which may require them to use increasing amounts of insulin throughout the day to maintain their blood sugar level or cause them to experience unpredictable responses to food or insulin.

Type 2 diabetes is a chronic condition where the body resists the effects of insulin and/or does not produce enough insulin to maintain normal glucose levels. Type 2 diabetes is influenced by environmental factors (diet and exercise) as well as by genetics, although its exact cause is unknown. It is a prominent disease in the US; a staggering 34.2 million people are diagnosed and another 88 million are considered pre-diabetic, according to the CDC's 2020 National Diabetes Statistics Report. The health costs related to diabetes in 2017 were $327 billion USD and have been steadily increasing. Due to these factors, there is a growing effort to identify cases of pre-diabetes in order to reduce the number of new cases of Type 2 diabetes to curb this massive public health problem and dependency on injectable insulin. Over consumption of sugar and an improper insulin response are trademarks of Type 2 diabetes. Investigation of therapeutics with the ability to resist drastic blood glucose spikes while also helping maintain proper insulin levels is an important step to addressing this disease.

Metabolic syndrome is a cluster of conditions that occur together, increasing the risk of heart disease, stroke and type 2 diabetes. These conditions include increased blood pressure, high blood sugar, excess body fat around the waist, and abnormal cholesterol or triglyceride levels.

Chronically elevated blood sugar due to impaired glucose metabolism can also contribute to fatty liver diseases, including non-alcoholic fatty liver disease (NAFLD), non-alcoholic steatohepatitis (NASH), and alcohol-related fatty liver disease (ALD).

Like humans, dogs and cats are experiencing higher rates of obesity and diabetes. The 2018 obesity rate in dogs and cats was reported to be 18.9 and 33.8 percent, respectively. Dogs in particular are prone to metabolic syndrome, which is associated with elevated blood glucose levels. Pet owners are in need of practical and effective treatment options to help manage their pet's diabetes and glucose levels.

Faecalibacterium prausnitzii (FP) is a commensal bacterium naturally occurring in the gastrointestinal tract of birds and mammals. WO2013130624A2, incorporated herein by reference, describes methods of using Faecalibacterium prausnitzii to improve weight gain, provide prophylaxis against diarrhea and improve feed efficiency in animals. WO2018118783A1, incorporated herein by reference, describes methods of using Faecalibacterium prausnitzii to improve milk production in animals, e.g., cattle. WO2018236979A1, incorporated herein by reference, describes methods of using Faecalibacterium prausnitzii and compositions derived from culture of Faecalibacterium prausnitzii to prevent or decrease growth of other microorganisms, particularly pathogenic organisms.

Improved methods for treatment of diseases and conditions characterized by impaired glucose metabolism are needed.

### SUMMARY

It has surprisingly been discovered that Faecalibacterium prausnitzii and compositions made from culture of Faecalibacterium prausnitzii, e.g., a composition (hereinafter referred to as “FPZ”) comprising materials derived from one or more Faecalibacterium prausnitzii cultures, including live cells, killed cells, cell components, and/or supernatant from such cultures, demonstrates utility in the treatment of diseases and conditions characterized by impaired glucose metabolism, such as Type 2 diabetes and pre-diabetic conditions, thereby reducing the need for exogenous insulin and other anti-diabetic drugs and for frequent blood glucose monitoring, and delaying or preventing the progression from pre-diabetic conditions to Type 2 diabetes.

For example, in mouse models, as described in the Examples, FPZ significantly increases insulin sensitivity and glucose tolerance in pre-diabetic and diabetic diet induced obese mice (C57BL/6J DIO). Moreover, the benefits are not merely acute, but are also disease-modifying, so that the treated mice exhibit a long-term improvement of insulin sensitivity compared to non-treated mice. Finally, although FPZ enhances insulin sensitivity and glucose tolerance in pre-diabetic and diabetic diet induced obese mice (C57BL/6J DIO), it does not lead to hypoglycemia in non-diabetic mice, and is thus believed to have a safety advantage over conventional anti-diabetic drugs and to be safe for use as a food supplement in a population having varying levels of baseline insulin sensitivity and glucose tolerance.

The disclosure therefore provides, in a first embodiment, a method of prophylaxis, treatment, or mitigation of a disease or condition characterized by impaired glucose metabolism, e.g., selected from Type 2 diabetes, Type 1 diabetes with insulin resistance, pre-diabetic conditions, insulin resistance, metabolic syndrome, or a fatty liver disease (e.g., non-alcoholic fatty liver disease (NAFLD), nonalcoholic steatohepatitis (NASH), and alcohol-related fatty liver disease (ALD)), comprising administering an effective amount of FPZ to a subject in need thereof.

The disclosure provides, in another embodiment, a composition comprising FPZ, e.g., a pharmaceutical composition, nutritional supplement, or food additive, e.g., wherein the embodiment is a liquid solution or dried, e.g. lyophilized.

The disclosure provides, in another embodiment, a method of making FPZ-S, e.g., a mixture of cells and supernatant from FP strains, comprising culturing a strain of Faecalibacterium prausnitzii, centrifuging the Faecalibacterium culture, to separate it into a supernatant portion and a sediment portion, and drying the product, for example, comprising one or more of the following steps:
a. Culturing Faecalibacterium prausnitzii;
b. Optionally killing the Faecalibacterium prausnitzii, e.g., by exposing to oxygen;
c. Centrifuging the optionally killed Faecalibacterium prausnitzii culture, to separate it into a supernatant portion and a sediment portion;
d. Removing excess water from the supernatant portion, e.g., using reverse osmosis
e. Combining the product of step (d) with the sediment portion;
f. Drying the product of step (e) to obtain a powder, e.g., by lyophilization;
g. Optionally mixing the powder thus produced with one or more diluents or carriers, or with a food or a beverage.

Further areas of applicability of the present disclosure will become apparent from the detailed description provided hereinafter. The detailed description and specific examples, while indicating the preferred embodiment of the invention, are intended for purposes of illustration only and are not intended to limit the scope of the invention.

### DESCRIPTION OF FIGURES

FIG. 1A depicts blood glucose measurements during GTT after 7 day FPZ-S treatment of C57BL/6J DIO prediabetic mice; FIG. 1B depicts blood glucose measurements during GTT after 14 day FPZ-S treatment of C57BL/6J DIO diabetic mice; and FIG. 1C depicts blood glucose measurements during GTT after 10 day FPZ-S treatment of five month-old C57BL/6J DIO mice.

FIG. 2 depicts the area under curve (AUC) calculated for glucose tolerance tests without using a baseline for mice pretreated with FPZ-S at 11.1 weeks (7 days treatment), 18.3 weeks (14 days treatment), and 23.4 weeks (10 days treatment).

FIG. 3 depicts the effect of three FPZ formulations (FPZ-S, a mixture of killed cells and supernatant from FP strains, FPZ-4, a mixture of killed cells and supernatant from one FP strain, and FPZ-L, a mixture of live cells and supernatant from FP strains) on glucose tolerance in DIO mice. C57BL/6J DIO mice are treated with three formulations of FPZ at 38 weeks of age for 14 days. Following treatment, mice are fasted for 16 h followed by a glucose tolerance test with blood glucose levels measured over 2 hours.

FIG. 4 depicts the effect of three FPZ formulations on % A1c levels in DIO mice. A) Hb A1c after 30 days of treatment of 38-week-old mice. B) Comparison of Hb A1c level before (week 34) and after (week 42) treatment with different formulations of FPZ.

FIG. 5 depicts that FPZ does not lead to hypoglycemia in non-diabetic mice. Mice treated with FPZ show comparable fasting glucose and similar response during a glucose tolerance test versus non-treated mice with both glucose spike and area under the curve not differing statistically.

FIG. 6 depicts that treatment with FPZ formulations does not lead to hypoglycemia in previously obese mice converted to normal diet. In mice that have been switched from high fat to normal diets, levels of A) fasting blood glucose and B) Percent A1c are not significantly reduced in mice treated with three formulations of FPZ versus control, indicating that while FPZ reduces glucose levels in DIO mice, it does not lead to hypoglycemia in non-diabetic mice.

FIG. 7 depicts the fasting blood glucose level in C57BL/6J mice after 2 weeks of treatment with FPZ, metformin, or a combination of FPZ with metformin as compared to non-treated (control) mice.

FIG. 8 depicts the fasting blood glucose level in C57BL/6J mice after 7 weeks of treatment with FPZ, metformin, or a combination of FPZ with metformin as compared to non-treated (control) mice.

### DESCRIPTION

In a first embodiment, the disclosure provides a method (Method 1) for propylaxis, treatment or mitigation of a disease or condition characterized by impaired glucose metabolism, e.g., selected from Type 2 diabetes, Type 1 diabetes with insulin resistance, pre-diabetic conditions, insulin resistance, metabolic syndrome, or a fatty liver disease (e.g., non-alcoholic fatty liver disease (NAFLD), nonalcoholic steatohepatitis (NASH), and alcohol-related fatty liver disease (ALD), comprising administering an effective amount of FPZ, to a subject in need thereof, for example:
1.1. Method 1, wherein the subject is a human.
1.2. Method 1, wherein the subject is a companion animal, e.g., a dog or cat.
1.3. Method 1, wherein the subject is diabetic.
1.4. Method 1, wherein the subject is pre-diabetic.
1.5. Method 1, wherein the subject has metabolic syndrome.
1.6. Method 1 wherein the subject has normal fasting blood glucose levels or wherein the subject has elevated fasting blood glucose levels.
1.7. Method 1 wherein the subject is overweight, e.g., wherein the subject is a human with a body-mass index (BMI) of over 25, e.g. a BMI of 30 or more.
1.8. Any foregoing method wherein the subject has an Hb A1C level of 5.7 percent or higher.
1.9. Any foregoing method wherein the subject has an Hb A1C level of 6.4 percent or higher.
1.10. Any foregoing method wherein the subject has a blood glucose level of 200 milligrams per deciliter (mg/dL) or higher.
1.11. Any foregoing method wherein the subject has a fasting blood glucose level of 100 milligrams per deciliter (mg/dL) or higher.
1.12. Any foregoing method wherein the subject has a fasting blood glucose level of greater than 125 milligrams per deciliter (mg/dL).
1.13. Any foregoing method wherein the FPZ is derived from a strain of Faecalibacterium prausnitzii that exhibits elevated production of butyrate, e.g. relative to a control strain, e.g., relative to reference strain DSM 17677.
1.14. Any foregoing method wherein the FPZ increases levels of IL-10 and/or IL-12 and/or reduces levels of IL-17 in mammalian cell culture, e.g., in peripheral blood mononuclear cell (PBMC) culture or in primary splenocyte and bone marrow-derived dendritic cell (BMDC) culture, relative to baseline or untreated cell culture.
1.15. Any foregoing method wherein the strain of Faecalibacterium prausnitzii used to make the FPZ is selected based on its effect, or the effect of FPZ made therefrom, in increasing levels of IL-10 and/or IL-12 and/or reducing levels of IL-17 in mammalian cell culture, e.g., in peripheral blood mononuclear cell (PBMC) culture or in primary splenocyte and bone marrow-derived dendritic cell (BMDC) culture, relative to baseline or untreated cell culture.
1.16. Any foregoing method wherein the Faecalibacterium prausnitzii used to make the FPZ is cultured in media free of any animal derived components comprising optimized mixture of nitrogen and carbon sources, and other nutritional components, including peptides, amino acids, carbohydrates, minerals, vitamins, and salts.
1.17. Any foregoing method wherein the Faecalibacterium prausnitzii used to make the FPZ has a 16S rRNA gene sequence comprising a sequence selected from GenBank (NCBI) accession numbers KJ957841 to KJ957877.
1.18. Any foregoing method wherein the subject is also receiving one or more anti-diabetic drugs, e.g., selected from metformin, sulfonylureas (e.g. glyburide, glipizide, or glimepiride), meglitinides (e.g. repaglinide or nateglinide), thiazolidinediones (e.g., rosiglitazone or pioglitazone), DPP-4 inhibitors (e.g., sitagliptin, saxagliptin, or linagliptin), GLP-1 receptor agonists (e.g., exenatide, liraglutide, or semaglutide), SGLT2 inhibitors (e.g., canagliflozin, dapagliflozin or empagliflozin).
1.19. Any foregoing method wherein the subject eliminates or reduces the dosage of one or more of anti-diabetic drugs, e.g., selected from metformin, sulfonylureas (e.g. glyburide, glipizide, or glimepiride), meglitinides (e.g. repaglinide or nateglinide), thiazolidinediones (e.g., rosiglitazone or pioglitazone), DPP-4 inhibitors (e.g., sitagliptin, saxagliptin, or linagliptin), GLP-1 receptor agonists (e.g., exenatide, liraglutide, or semaglutide), SGLT2 inhibitors (e.g., canagliflozin, dapagliflozin or empagliflozin) during or consequent to the treatment.
1.20. Any foregoing method wherein the subject is also receiving insulin or an insulin analog (e.g., insulin lispro, insulin aspart, insulin glulisine, insulin detemir, insulin degludec, or insulin glargine).
1.21. Any foregoing method wherein the subject eliminates or reduces the dosage of insulin or an insulin analog (e.g., insulin lispro, insulin aspart, insulin glulisine, insulin detemir, insulin degludec, or insulin glargine) during or consequent to the treatment.
1.22. Any foregoing method wherein the subject suffers from Type 1 diabetes with insulin resistance and is receiving insulin or an insulin analog (e.g., insulin lispro, insulin aspart, insulin glulisine, insulin detemir, insulin degludec, or insulin glargine), and the dosage of one or more of insulin or an insulin analog is reduced during or consequent to the treatment.
1.23. Any foregoing method wherein the subject is receiving one or more of blood thinners (e.g., selected from aspirin, coumarins and indandiones, factor Xa inhibitors, heparins, or thrombin inhibitors), blood pressure medications (e.g., selected from angiotensin-converting enzyme (ACE) inhibitors, diuretics, angiotensin II receptor blockers (ARBs), Calcium channel blockers, beta blockers, and renin inhibitors), and statins (e.g., selected from atorvastatin, fluvastatin, lovastatin, pitavastatin, pravastatin, rosuvastatin, and simvastatin).
1.24. Any foregoing method wherein the subject's Hb A1C level is reduced relative to the subject's Hb A1C level at the start of the treatment.
1.25. Any foregoing method wherein the subject's fasting blood glucose level is reduced relative to the subject's fasting blood glucose level at the start of treatment.
1.26. Any foregoing method wherein the treatment enhances insulin sensitivity in the subject.
1.27. Any foregoing method wherein the treatment enhances insulin release in the subject in response to glucose challenge.
1.28. Any foregoing method wherein the subject has a pre-diabetic condition or metabolic syndrome, and the method inhibits progression of the pre-diabetic condition or metabolic syndrome to Type 2 diabetes.
1.29. Any foregoing method wherein the effective amount of FPZ is administered once daily.
1.30. Any foregoing method wherein the effective amount FPZ is administered daily for at least one week, e.g., for at least one month, e.g., for at least three months.
1.31. Any foregoing method wherein the FPZ comprises dried cells, cell components, and supernatant from a Faecalibacterium prausnitzii culture, e.g., a composition according to any one of Composition 1, et seq.
1.32. Any foregoing method wherein the FPZ comprises an extract from a culture of Faecalibacterium prausnitzii.
1.33. Any foregoing method wherein the FPZ comprises live cells of Faecalibacterium prausnitzii.
1.34. Any foregoing method wherein the FPZ comprises killed cells of Faecalibacterium prausnitzii, e.g., wherein the Faecalibacterium prausnitzii has been killed by exposure to oxygen.
1.35. Any foregoing method wherein the FPZ comprises cell components of Faecalibacterium prausnitzii.
1.36. Any foregoing method wherein the FPZ comprises supernatant from a culture of
Faecalibacterium prausnitzii.
1.37. Any foregoing method wherein the FPZ is made from or comprises materials from two or more different strains of Faecalibacterium prausnitzii.
1.38. Any foregoing method wherein the FPZ comprises a supernatant from one or more Faecalibacterium prausnitzii cultures wherein the supernatant is enriched for molecules by fractionation (e.g., separated by molecular weight, separated by charge, and/or separated by hydrophobicity).
1.39. Any foregoing method wherein the FPZ is in the form of a dry powder, e.g., a lyophosate, e.g., a lyophosate prepared from cells, cell components, and supernatant from a Faecalibacterium prausnitzii culture.
1.40. Any foregoing method wherein the FPZ is mixed with food.
1.41. Any foregoing method wherein the FPZ is mixed with liquid, e.g., mixed with water or mixed with a beverage.
1.42. Any foregoing method wherein the FPZ is admixed with a pharmaceutically acceptable diluent or carrier, e.g., is in the form of a tablet, capsule or powder.
1.43. Any foregoing method wherein the FPZ is administered in a daily dosage.
1.44. Any foregoing method wherein the FPZ is administered ad libitum.
1.45. Any foregoing method wherein the treatment is intermittent, e.g., daily administration for up to 15 days, e.g., 5-10 days, followed by a period, e.g., up to six months, e.g., one to three months, without treatment, followed by a second period of daily administration for up to 15 days, e.g., 5-10 days.
1.46. Any foregoing method wherein the treatment has a disease-modifying effect; for example wherein fasting blood glucose levels are reduced relative to baseline even after treatment has ceased.
1.47. Any foregoing method wherein the subject has a genetic predisposition to develop Type 2 diabetes, e.g., wherein the subject is a human who has a genetic polymorphism associated with Type 2 diabetes, e.g., wherein the subject exhibits a polymorphism associated with Type 2 diabetes in one or more of the following genes: TCF7L2, PPARG, FTO, KCNJ11, NOTCH2, WFS1, IGF2BP2, SLC30A8, JAZF1, HHEX, DGKB, CDKN2A, CDKN2B, KCNQ1, HNF1A, HNF1B, MC4R, GIPR, HNF4A, MTNR1B, PARG6, ZBED3, SLC30A8, CDKAL1, GLIS3, GCK, and GCKR.
1.48. Any foregoing method wherein the disease or condition characterized by impaired glucose metabolism is Type 2 diabetes.
1.49. Any foregoing method wherein the disease or condition characterized by impaired glucose metabolism is Type 1 diabetes with insulin resistance.
1.50. Any foregoing method wherein the disease or condition characterized by impaired glucose metabolism is a pre-diabetic condition.
1.51. Any foregoing method wherein the disease or condition characterized by impaired glucose metabolism is insulin resistance.
1.52. Any foregoing method wherein the disease or condition characterized by impaired glucose metabolism is metabolic syndrome.
1.53. Any foregoing method wherein the disease or condition characterized by impaired glucose metabolism is a fatty liver disease.
1.54. Any foregoing method wherein the disease or condition characterized by impaired glucose metabolism is non-alcoholic fatty liver disease (NAFLD).
1.55. Any foregoing method wherein the disease or condition characterized by impaired glucose metabolism is nonalcoholic steatohepatitis (NASH).
1.56. Any foregoing method wherein the disease or condition characterized by impaired glucose metabolism is alcohol-related fatty liver disease (ALD).
1.57. Any foregoing method wherein the administration of FPZ to a subject having normal blood glucose levels does not result in hypoglycemia.
1.58. Any foregoing method wherein the FPZ is administered as a food supplement.
1.59. Any foregoing method wherein the FPZ is administered to a population having a normal range of fasting blood glucose levels.
1.60. Any foregoing method which is a method of prophylaxis in a subject having normal fasting blood glucose levels and/or normal Hb A1C, e.g., wherein the subject is at elevated risk of developing a disease or condition characterized by impaired glucose metabolism, e.g., a disease or condition selected from Type 2 diabetes, Type 1 diabetes with insulin resistance, pre-diabetic conditions, insulin resistance, metabolic syndrome, or a fatty liver disease (e.g., non-alcoholic fatty liver disease (NAFLD), nonalcoholic steatohepatitis (NASH), and alcohol-related fatty liver disease (ALD).

The disclosure provides, in another embodiment, a composition (Composition 1) comprising optionally dried cells, cell components, and supernatant from a Faecalibacterium prausnitzii culture:
1.1. Composition 1 wherein a Faecalibacterium prausnitzii culture has been centrifuged to separate it into a supernatant portion and a sediment portion, which are then recombined.
1.2. Any foregoing composition wherein the Faecalibacterium prausnitzii culture is killed, e.g., by exposure to oxygen, then centrifuged to separate it into a supernatant portion and a sediment portion.
1.3. Any foregoing composition wherein the Faecalibacterium prausnitzii cells, cell components, and supernatant are dried.
1.4. Any foregoing composition wherein the Faecalibacterium prausnitzii cells, cell components, and supernatant are lyophilized.
1.5. Any foregoing composition which is suitable for oral administration to a human.
1.6. Any foregoing composition which is suitable for oral administration to a companion animal, e.g., to a dog or cat.
1.7. Any foregoing composition which is a pharmaceutical composition, e.g., a tablet, capsule, or powder, e.g., comprising Faecalibacterium prausnitzii cells, cell components and supernatant, in combination or association with one or more pharmaceutical diluents or carriers.
1.8. Any foregoing composition which is an enteric-coated tablet or capsule comprising Faecalibacterium prausnitzii cells and cell components and supernatant, e.g., wherein the Faecalibacterium prausnitzii cells and cell components and supernatant are in dried or lyophilized form.
1.9. Any foregoing composition which is a food or beverage.
1.10. Any foregoing composition which is a food or beverage mixed with a composition comprising Faecalibacterium prausnitzii cells, cell components, and supernatant.
1.11. Any foregoing composition wherein the Faecalibacterium prausnitzii is a strain that exhibits elevated production of butyrate, e.g., relative to a control strain, e.g., relative to reference strain DSM 17677.
1.12. Any foregoing composition wherein the Faecalibacterium prausnitzii comprises a combination of two or more strains.
1.13. Any foregoing composition wherein the composition increases levels of IL-10 and/or IL-12 and/or reduces levels of IL-17 in mammalian cell culture, e.g., in peripheral blood mononuclear cell (PB MC) culture or in primary splenocyte and bone marrow-derived dendritic cell (BMDC) culture, relative to baseline or untreated cell culture.
1.14. Any foregoing composition wherein the Faecalibacterium prausnitzii strain is selected based on its effect in increasing levels of IL-10 and/or IL-12 and/or reducing levels of IL-17 in mammalian cell culture, e.g., in peripheral blood mononuclear cell (PBMC) culture or in primary splenocyte and bone marrow-derived dendritic cell (BMDC) culture, relative to baseline or untreated cell culture.
1.15. Any foregoing composition which is effective for propylaxis, treatment or mitigation of a disease or condition characterized by impaired glucose metabolism, e.g., a disease or condition selected from Type 2 diabetes, Type 1 diabetes with insulin resistance, pre-diabetic conditions, insulin resistance, metabolic syndrome, or a fatty liver disease (e.g., non-alcoholic fatty liver disease (NAFLD), nonalcoholic steatohepatitis (NASH), and alcohol-related fatty liver disease (ALD), e.g., which is effective in any of Method 1, et seq.
1.16. Any foregoing composition wherein the Faecalibacterium prausnitzii is cultured in media free of any animal-derived components comprising optimized mixture of nitrogen and carbon sources, and other nutritional components, including peptides, amino acids, carbohydrates, minerals, vitamins, and salts.
1.17. Any foregoing composition wherein the Faecalibacterium prausnitzii has a 16S rRNA gene sequence comprising a sequence selected from GenBank (NCBI) accession numbers KJ957841 to KJ957877.
1.18. Any foregoing composition for use in any of Methods 1, et seq.
1.19. Any foregoing composition which is obtained or obtainable by the steps of:
   a. Culturing Faecalibacterium prausnitzii;
   b. Optionally killing the Faecalibacterium prausnitzii, e.g., by exposing to oxygen;
   c. Centrifuging the optionally killed Faecalibacterium prausnitzii culture, to separate it into a supernatant portion and a sediment portion;
   d. Removing excess water from the supernatant portion, e.g., using reverse osmosis;
   e. Combining the product of step (d) with the sediment portion;
   f. Drying the product of step (e) to obtain a powder, e.g., using lyophilization; and
   g. Optionally combining the powder thus produced with one or more diluents or carriers, or with a food or a beverage.
1.20. Any foregoing composition which is obtained or obtainable by the steps of:
   a. Culturing Faecalibacterium prausnitzii;
   b. Killing the Faecalibacterium prausnitzii by exposing it to oxygen;
   c. Centrifuging the killed Faecalibacterium prausnitzii culture, to separate it into a supernatant portion and a sediment portion;
   d. Removing excess water from the supernatant portion using reverse osmosis;
   e. Combining the product of step (d) with the sediment portion;
   f. Drying the product of step (e) to obtain a powder using lyophilization; and
   g. Combining the powder thus produced with one or more diluents or carriers, or with a food or a beverage.

The disclosure further provides the use of Faecalibacterium prausnitzii, or a composition made from a culture of Faecalibacterium prausnitzii, e.g., an extract from a culture of Faecalibacterium prausnitzii, or a composition comprising Faecalibacterium prausnitzii cells, cell components and supernatant, or any FPZ or FPZ-S as described herein, e.g., a composition according to any of Composition 1, et seq., in the manufacture of a medicament for treating or mitigating a disease or condition characterized by impaired glucose metabolism, e.g., selected from Type 2 diabetes, Type 1 diabetes with insulin resistance, pre-diabetic conditions, insulin resistance, metabolic syndrome, and fatty liver disease (e.g., selected from non-alcoholic fatty liver disease (NAFLD), nonalcoholic steatohepatitis (NASH), and alcohol-related fatty liver disease (ALD)), e.g., in accordance with any of Method 1, et seq.

The disclosure further provides Faecalibacterium prausnitzii, or a composition made from a culture of Faecalibacterium prausnitzii, e.g., FPZ, e.g., an extract from a culture of Faecalibacterium prausnitzii, or a composition comprising Faecalibacterium prausnitzii cells, cell components and supernatant, e.g. a composition according to any of Composition 1, et seq., for use in treating or mitigating a disease or condition characterized by impaired glucose metabolism, selected from Type 2 diabetes, Type 1 diabetes with insulin resistance, pre-diabetic conditions, insulin resistance, metabolic syndrome, and fatty liver disease (e.g., selected from non-alcoholic fatty liver disease (NAFLD), nonalcoholic steatohepatitis (NASH), and alcohol-related fatty liver disease (ALD)), e.g., in accordance with any of Method 1, et seq.

In some embodiments, the methods herein use live Faecalibacterium prausnitzii cells. In some embodiments, the disclosure use killed Faecalibacterium prausnitzii cells. In some embodiments, the disclosure use supernatant of Faecalibacterium prausnitzii. In some embodiments, the disclosure use killed cells and supernatant of Faecalibacterium prausnitzii, e.g., wherein the killed cells and supernatant of Faecalibacterium prausnitzii are dried, e.g., lyophilized.

In some embodiments, the strains of Faecalibacterium prausnitzii used exhibit relatively high butyrate production, e.g., as measured using gas chromatography of culture supernatant indicating a concentration of butyrate exceeding 1000 ppm. For example, Faecalibacterium prausnitzii isolates and a reference strain, DSM 17677, are inoculated in 25 ml of nutrient broth and incubated at 37° C. for 48 h under anaerobic conditions. The culture is centrifuged, the supernatant is collected, and the concentration of acetate, butyrate, propionate and isobutyrate in the media before inoculation and in the supernatant of the culture was measured by gas chromatography. Samples are injected into a gas chromatograph, and the analysis is performed according to the manufacturer's protocol. Isolates producing relatively high levels of butyrate in the supernatant, e.g., greater than the reference strain, e.g., at least 1000 ppm, are selected.

In a further embodiment, the disclosure provides a method of making a composition, e.g., of Composition 1, et seq. comprising optionally dried cells and cell components and supernatant from a Faecalibacterium prausnitzii culture, comprising culturing the Faecalibacterium prausnitzii, centrifuging the Faecalibacterium culture, to separate it into a supernatant portion and a sediment portion, and drying the product, e.g., comprising the following steps:

a. Culturing the Faecalibacterium;
b. Optionally killing the Faecalibacterium, e.g., by exposing to oxygen;
c. Centrifuging the optionally killed Faecalibacterium prausnitzii culture, to separate it into a supernatant portion and a sediment portion;
d. Removing excess water from the supernatant portion, e.g., using reverse osmosis and/or using drying, e.g., spray-drying or evaporation;
e. Combining the product of step (d) with the sediment portion;
f. Drying the product of step (e) to obtain a powder;
g. Optionally combining the powder thus produced with one or more diluents or carriers, or with a food or a beverage.

In a further embodiment, the disclosure provides Faecalibacterium prausnitzii, or a composition made from a culture of Faecalibacterium prausnitzii, e.g., FPZ, e.g., an extract from a culture of Faecalibacterium prausnitzii, or a composition comprising Faecalibacterium prausnitzii cells, cell components and supernatant, e.g., a composition according to any of Composition 1, et seq., in combination with one or more biguanide anti-diabetic agents, e.g., selected from buformin, phenformin, imeglimin, and metformin (e.g., metformin) for use in treating or mitigation a disease or condition characterized by impaired glucose metabolism, selected from Type 2 diabetes, Type 1 diabetes with insulin resistance, pre-diabetic conditions, metabolic syndrome, and fatty liver disease (e.g., selected from non-alcoholic fatty liver disease (NAFLD), nonalcoholic steatohepatitis (NASH), and alcohol-related fatty liver disease (ALD)), e.g., for use in accordance with any of Method 1, et seq.

For example, the invention provides a method of treating, inhibiting or mitigating a disease or condition characterized by impaired glucose metabolism, selected from Type 2 diabetes, Type 1 diabetes with insulin resistance, pre-diabetic conditions, metabolic syndrome, and fatty liver disease (e.g., selected from non-alcoholic fatty liver disease (NAFLD), nonalcoholic steatohepatitis (NASH), and alcohol-related fatty liver disease (ALD)) comprising administering a synergistically effective amount of (i) Faecalibacterium prausnitzii, or a composition made from a culture of Faecalibacterium prausnitzii, e.g., FPZ, e.g., an extract from a culture of Faecalibacterium prausnitzii, or a composition comprising Faecalibacterium prausnitzii cells, cell components and supernatant, e.g., a composition according to any of Composition 1, et seq., in combination with (ii) one or more biguanide anti-diabetic agents, e.g., selected from buformin, phenformin, imeglimin, and metformin (e.g., metformin), e.g., in accordance with any of Method 1, et seq.

In another embodiment, the invention provides a method of reducing the effective dose of a drug for treating, inhibiting or mitigating a disease or condition characterized by impaired glucose metabolism, for example reducing the effective dose of a drug selected from biguanide anti-diabetic agents, e.g., selected from buformin, phenformin, imeglimin, and metformin (e.g., metformin), comprising administering a synergistically effective amount of (i) Faecalibacterium prausnitzii, or a composition made from a culture of Faecalibacterium prausnitzii, e.g., FPZ, e.g., an extract from a culture of Faecalibacterium prausnitzii, or a composition comprising Faecalibacterium prausnitzii cells, cell components and supernatant, e.g., a composition according to any of Composition 1, et seq., together with a reduced dose of the drug, e.g., wherein the drug is metformin and the reduced daily dosage of the drug is less than 2000 mg, e.g., less than 1000 mg, e.g., 500 mg-1000 mg.

In some embodiments, the disclosure provides a method for prophylaxis, treatment, or mitigation of a condition characterized by muscle atrophy induced by an anti-diabetic drug (e.g., metformin) comprising administering an effective amount of a combination of FPZ and optionally continuing to administer the anti-diabetic drug, (e.g., metformin), to a subject in need thereof.

### Example 1: Glucose Tolerance Test in C57BL/6J Mice Using Faecalibacterium prausnitzii Killed Cell Component and Supernatant

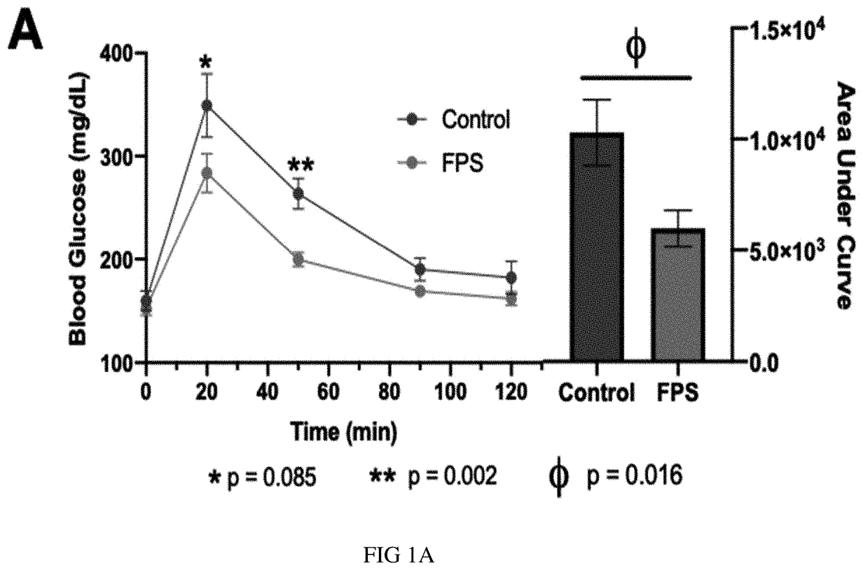

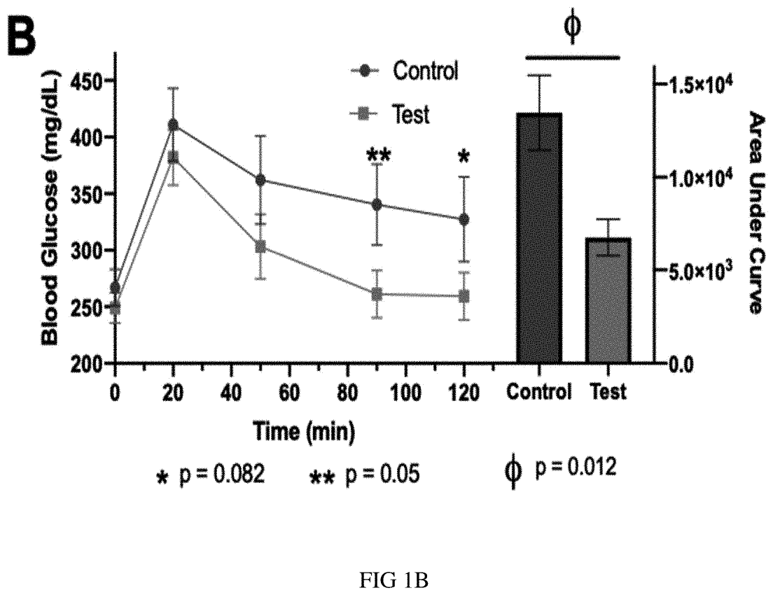

Three mouse trials are performed to assess whether FPZ-S is able to show the same positive effects in controlling glucose metabolism, with the results shown in FIG. 1. The study uses C57BL/6J mice, which are prediabetic and known to develop a type 2 diabetic phenotype after long-term feeding of a high fat diet. Prior to the initiation of treatment, mice receive a high fat diet (60% fat) ad libitum to induce the obesity phenotype with the high fat diet continued throughout the study. The obese mice have a fasting blood glucose between 100 and 200 mg/dL at the start of the trial. The mice are divided into control and treatment groups, and they are treated daily for 7 days with either control media or 1 mg/kg reconstituted FPZ-S, given by oral gavage. After 7 days, fasting blood glucose levels are determined and mice undergo a glucose tolerance test with the results shown in FIG. 1A. Mice treated with FPZ-S show significantly reduced blood glucose levels over the two hours following glucose administration (AUC, p=0.016) indicating that FPZ-S improves glucose tolerance. The second trial tests the effect of a 14 day treatment with reconstituted FPZ-S. The same mice from the 7 day trial are used and have developed Type 2 diabetes (fasting glucose>200 mg/dL) with the results shown in FIG. 1B. Similar to the results seen in the 7 day trial, the FPZ-S-treated mice with Type 2 diabetes show a reduction of blood glucose for two hours after a glucose tolerance test (AUC, p=0.012). FPZ-S treated mice are able to achieve baseline blood glucose levels by the end of the two-hour glucose tolerance test, while blood glucose levels in untreated control mice remained elevated. In a third trial, the same C57BL/6J DIO mice, at five months of age, are treated with FPZ-S for ten days. Similar to the other two trials, FPZ-S treated mice show significant reduction in blood glucose levels two hours after glucose administration (p=0.0035), as depicted in FIG. 1C.

Treatment with lyophilized FPZ-S of prediabetic and Type 2 diabetes model mice: 14 diet-induced obese C57BL/6J male mice (The Jackson Laboratory https://www.jax.org/strain/000664), are enrolled in the study at two months of age. Prior to the initiation of treatment, mice receive a high fat diet (60% fat, Research Diets Inc.) ad libitum to induce the obesity phenotype. The high fat diet is continued throughout the study. Mice are randomly allocated into one of two groups; placebo treatment (Control) and reconstituted lyophilized FPZ-S treatment. Upon enrollment at T-12 days, mice undergo an acclimation process by daily handling and daily administration of blank gavage. At Day 0, weight is recorded and treatment initiated. A daily oral dose of either treatment (FPZ-S, n=7) or control (Control, n=7) is administered for 7 days. After overnight fasting following the last treatment, mice undergo glucose tolerance (GTT) tests. A second trial is carried out using 11 four-month old C57BL/6J DIO mice (Control, n=5 and Test n=6) that have developed Type 2 diabetes (fasting blood glucose>200 mg/dL). Mice are treated with FPZ-S for 14 days followed by a GTT. A third trial is carried out on 14 five-month old C57BL/6J DIO mice, with 10 days of treatment.

FPZ-S treatment significantly and dramatically improves glucose tolerance in this diet induced obesity model mice trial. After seven days of treatment, FPZ-S-treated mice have lower glucose AUC after oral glucose administration (p=0.016) compared to controls, as shown in FIG. 1A. After 14 days of treatment, the FPZ-S-treated mice have lower glucose AUC after oral glucose administration (p=0.012) compared to controls, as shown in FIG. 1B. Blood glucose levels in FPZ-S treated mice also return to baseline levels within two hours after the start of the GTT, while the blood glucose levels of untreated mice remain elevated after 2 hours. Similarly, in the five-month old mice, after 10 days of treatment, the FPZ-S-treated mice have lower glucose AUC after oral glucose administration (p=0.0035) compared to control, as shown in FIG. 1C. A significantly lower level of blood glucose is also seen after fasting and during every time point after glucose administration.

### Example 2: Disease-Modifying Effects of Treatment with FPZ

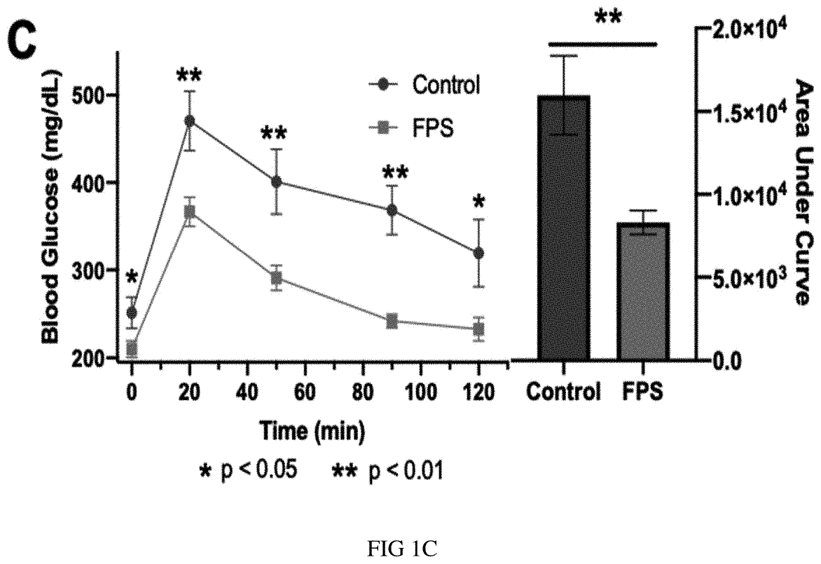

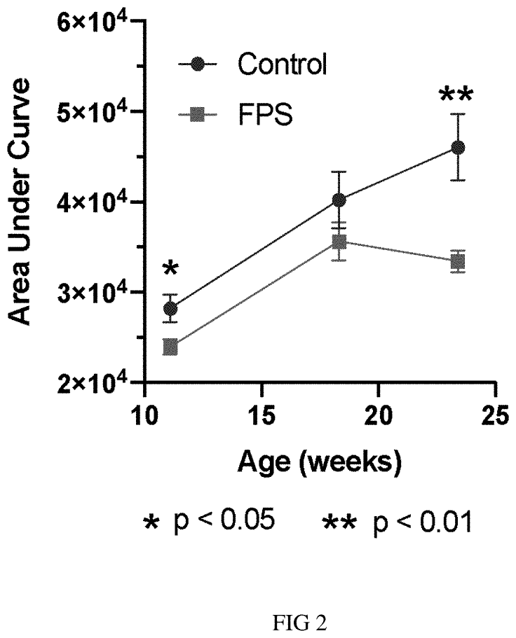

Diet-induced obese C57BL/6J male mice are given a high fat diet as described above to induce an obese phenotype and Type 2 diabetes-like disease. Test mice are pretreated with FPZ-S at 11.1 weeks (7 days treatment), 18.3 weeks (14 days treatment), and 23.4 weeks (10 days treatment), whereas controls are untreated. The area under curves (AUC) for blood levels of glucose are calculated for glucose tolerance tests without using a baseline and comparing treated mice with untreated controls, as depicted in FIG. 2. At all timepoints, the AUCs are lower for the treated mice than the untreated mice. The AUCs for the untreated control mice increase over time, indicating the mice are becoming less insulin-sensitive as they age. For the mice receiving intermittent FPZ-S treatment, however, after an initial increase, the AUCs decrease in mice after the second treatment of FPZ-S, suggesting that repeated treatments with FPZ-S, even on an intermittent basis, produce a long-term improvement of insulin sensitivity compared to non-treated mice.

### Example 3: Glucose Tolerance and % A1c Tests in C57BL/6J Mice with FPZ-S, FPZ-4 and FPZ-L

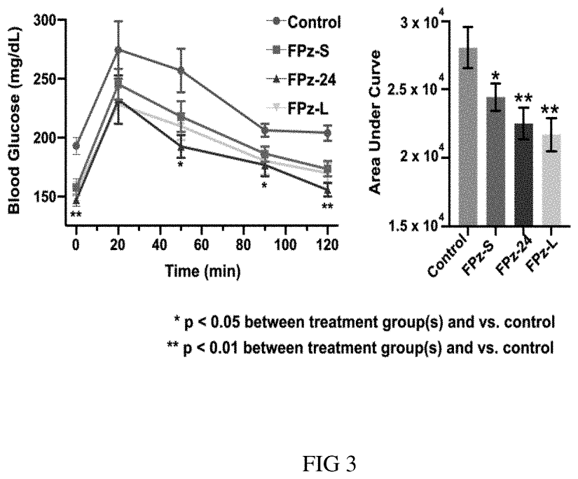

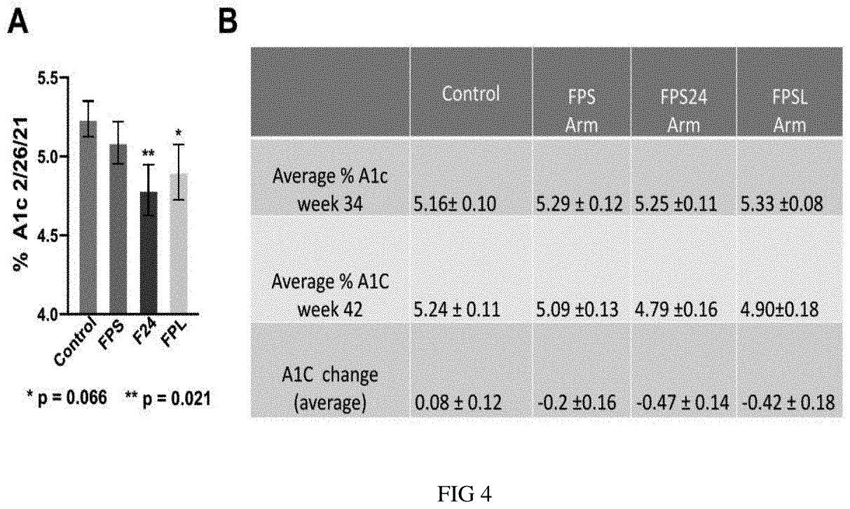

40-week-old mice are treated with the strain killed cell and supernatant mixture using in Example 1 (FPZ-S), live cell FPZ and supernatant (FPZ-L), and killed cell and supernatant from one strain of FPZ (FPZ-4). Mice in all treatment groups in Trial 2 display a significant difference in fasting blood glucose measurements following 14 days of treatment with FPZ products compared to CTL, as shown in FIG. 3. Following glucose administration, a significant decrease in blood glucose measurements compared to CTL were seen at 4 of 5 recorded timepoints. To investigate the changes in fasting blood glucose levels before and after treatment, measurements are taken before (34 weeks) and after treatment (40 weeks). All groups of treatment mice have lower average fasting glucose measurements after 14 days of treatment, while control is found to increase. Looking at Hb A1c, which corresponds to average blood glucose over the previous several weeks, FIG. 4A shows all mice in all treatment groups have lower Hb A1c than control 30 days after the start of treatment, with FPZ-4 and FPZ-L showing statistical significance. Looking at Hb A1c before and after treatment, FIG. 4B shows all treatment groups of mice experienced a decrease in Hb A1c values, while the control group shows an increase in Hb A1c.

### Example 4: Safe Treatment of Non-Diabetic Mice with FPZ

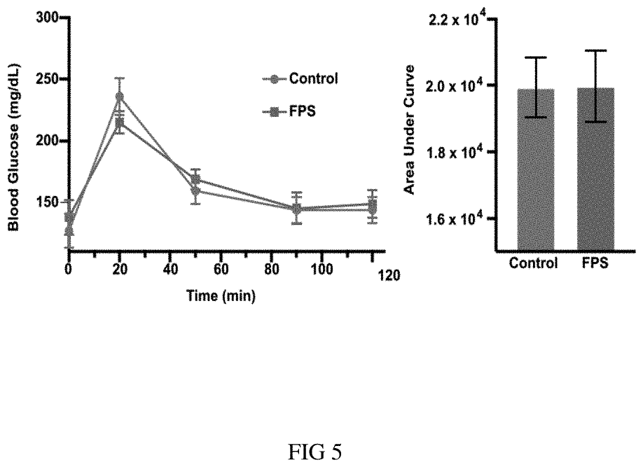

Male C57BL6/J mice are purchased from Jackson Laboratories and maintained on a standard chow diet for 14 days. Mice are separated into control and FPZ-S treatment groups. After 14 days of treatment, a GTT is performed and blood glucose levels measured. As shown in FIG. 5, mice treated with FPZ-S have comparable fasting glucose levels and show a similar GTT response as mice that are not treated with FPZ-S. The fact that these mice do not show reduced blood glucose levels demonstrates that FPZ-S is safe in healthy mice and does not result in hypoglycemia as seen with some other anti-diabetic therapies.

### Example 5: Safe Treatment of Previously Diabetic with FPZ

Male C57BL6/J mice are maintained on a high fat diet purchased from Research Diets for 46 weeks. Mice are then switched to a standard chow diet and maintained on this diet for 30 days. Mice are then administered the three FPZ formulations described above in the trial 2 summary for 28 days. Mice are then fasted for 16 h, fasting blood glucose levels are recorded. A GTT is carried out, blood glucose measurements are carried out, and blood glucose measurements are taken at 20, 50, 90, and 120 minute time points. Hb A1c levels are recorded immediately before diet change, immediately before commencement of treatment, and after the one-month treatment period. Weight is recorded twice a week for the duration of the trial.

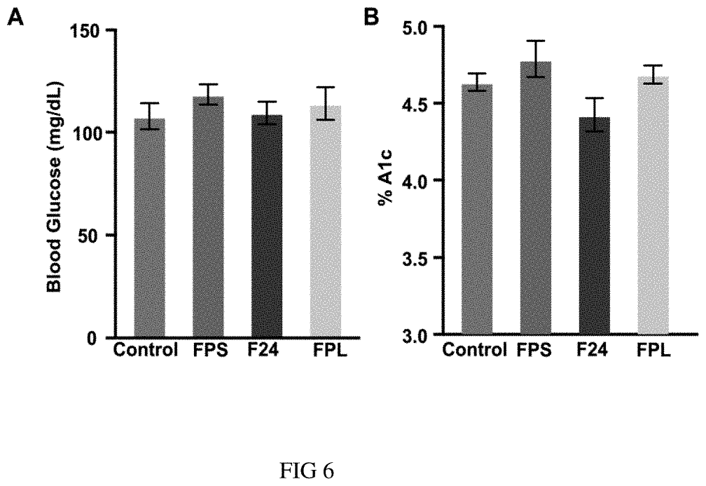

FIG. 6 shows that treatment with different FPZ formulations does not lead to hypoglycemia in previously obese mice converted to normal diet. In mice that have been switched from high fat to normal diets, levels of A) fasting blood glucose and B) percent Hb A1c are not significantly reduced in mice treated with three formulations of FPZ versus control, indicating that while FPZ reduces glucose levels significantly in diet-induced obese mice, it does not lead to hypoglycemia in non-diabetic mice.

### Example 6: Glucose Tolerance Test in C57BL/6J Mice with a Combination of Metformin and Faecalibacterium prausnitzii

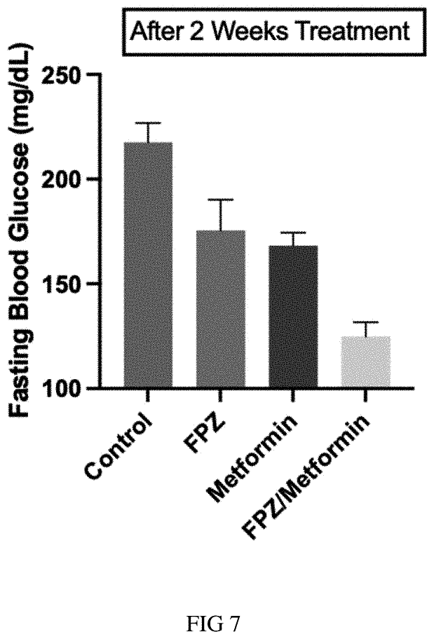

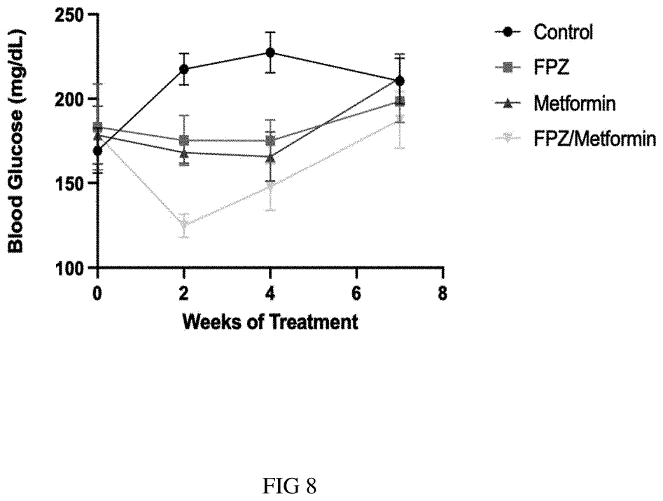

A mouse trial is performed to assess whether a combination of FPZ and metformin can show positive effects in controlling glucose metabolism, with the results shown in FIGS. 7 and 8. The study uses 29 C57BL/6J mice, which are placed on a high-fat diet and allowed to develop prediabetes. The mice are fasted for 16 h, and the fasting blood glucose levels are measured to be greater than 105 mg/dL. The mice are divided up into control and treatment groups, and they are treated daily for 7 weeks with either 100 mg/kg FPZ, 100 mg/kg metformin, or a combination of FPZ with metformin, given by oral gavage. Fasting blood glucose are recorded at 2, 4, and 7 weeks of treatment. As shown in FIG. 7, after 2 weeks of treatment, the FPZ and metformin groups show a 21% and 24% reduction in fasting blood glucose levels, respectively, compared to the control group, which are gavaged with water. For the mice group that is treated with a combination of FPZ and metformin, there is a 40% reduction in fasting blood glucose level compared to the control group, and a 19% reduction when compared to mice treated with metformin alone.

1. A method of prophylaxis, treatment or mitigation of a disease or condition characterized by impaired glucose metabolism, e.g., selected from Type 2 diabetes, Type 1 diabetes with insulin resistance, pre-diabetic conditions, insulin resistance, metabolic syndrome, or a fatty liver disease (e.g., non-alcoholic fatty liver disease (NAFLD), non-alcoholic steatohepatitis (NASH), and alcohol-related fatty liver disease (ALD)), comprising administering an effective amount of a composition (“FPZ”) comprising materials derived from one or more Faecalibacterium prausnitzii cultures, including live cells, killed cells, cell components, and/or supernatant from such cultures, e.g., comprising an extract from a culture of Faecalibacterium prausnitzii, or a composition comprising Faecalibacterium prausnitzii cells, cell components, and supernatant, to a subject in need thereof.

2. The method of claim 1 wherein the subject is a human or a companion animal.

3. The method of claim 1, wherein the FPZ does not cause a hypoglycemic effect in the subject.

4. The method of claim 1, which is a method of prophylaxis wherein the subject has normal fasting blood glucose and/or normal Hb A1c levels but is at elevated risk of developing a disease or condition characterized by impaired glucose metabolism.

5. The method of claim 1, wherein the subject has been diagnosed as being diabetic or pre-diabetic or as having metabolic syndrome.

6. The method of claim 1, wherein the FPZ comprises Faecalibacterium prausnitzii cells, cell components, and supernatant, wherein the Faecalibacterium prausnitzii cells, cell components, and supernatant have been dried, e.g., lyophilized.

7. The method of claim 1, wherein the FPZ is administered in the form of a pharmaceutical composition, nutritional supplement, or food or beverage comprising an effective amount of FPZ.

8. A composition comprising Faecalibacterium prausnitzii cells, cell components, and supernatant, e.g., a pharmaceutical composition, nutritional supplement, or food or beverage, e.g., wherein the Faecalibacterium prausnitzii cells, cell components, and supernatant have been dried, e.g. lyophilized.

9. A method of making a composition comprising Faecalibacterium prausnitzii cells, cell components and supernatant, comprising culturing the Faecalibacterium prausnitzii, centrifuging the Faecalibacterium prausnitzii culture, and drying the product.

10. The method of claim 1, wherein the subject is also receiving one or more anti-diabetic drugs, wherein the antidiabetic drug comprise metformin.
